(19) United States
(12) Patent Application Publication
(10) Pub. No.: US 2008/0108121 A1
(43) Pub. Date: May 8, 2008

(54) Method of arranging cells and electrode array applied thereto

(75) Inventors: Cheng-Hsien Liu, Hsinchu City (TW); Hwan-You Chang, Hsinchu City (TW); Long Hsu, Hsinchu City (TW); Chen-Ta Ho, Hsinchu City (TW); Ruei-Zeng Lin, Kaohsiung City (TW); Wai William Wang, Taoyuan City (TW); Ta-Yuan Lee, Linkou Township (TW); Chung-Cheng Chou, Lujhu Township (TW); Chen Peng, Taipei City (TW); Fung-Hsu Wu, Gueishan Township (TW)

Correspondence Address: RABIN & Berdo, PC, 1101 14TH STREET, NW, SUITE 500, WASHINGTON, DC, 20005, US

(73) Assignee: BenQ Corporation, Taoyuan Shien (TW)

(21) Appl. No.: 11/797,418

(22) Filed: May 3, 2007

(30) Foreign Application Priority Data
Nov. 3, 2006 (TW) 95140838

(51) Int. Cl. C12N 13/00; C12M 1/00

(52) U.S. Cl. 435/173.9; 435/283.1

(57) ABSTRACT

A method of arranging cells comprises: (a) applying a voltage to two electrodes so as to allow a plurality of cells suspended in a dielectrophoresis-manipulating buffer (DEP-manipulating buffer) to be driven to be arranged into a pattern; (b) replacing the DEP-manipulating buffer with a solution comprising calcium ion and magnesium ion which helps the patterned cells adhere to the substrate; and (c) replacing the solution comprising calcium ion and magnesium ion with a medium so as to allow the patterned cells to grow on the substrate.

This application claims the benefit of Taiwan application Serial No. 095140838, filed Nov. 3, 2006, the subject matter of which is incorporated herein by reference.

### BACKGROUND OF THE INVENTION

Field of the Invention

The invention relates in general to a method of arranging cells and an electrode array applied thereto, and more particularly to the method of arranging cells by dielectrophoresis and the electrode array applied thereto.

Description of the Related Art

The human liver is covered by a slim and compact membrane consisting of connective tissue, and the membrane projects into the liver and forms a netlike skeleton. The liver is morphologically divided into many units of similar shape and function, as so-called hepatic lobules, by the skeleton.

Referring to FIG. 1, schematically illustrating a cross sectional view of a classic hepatic lobule of human liver. A human liver is constructed by about 15 million hepatic lobules, which take the shape of irregular hexagonal prisms. The cross section of the hepatic lobule are a shape of hexagon and an area of 1×2 square millimeters. The hepatic lobule is filled with cords of liver parenchyma cells and hepatocytes, which radiate from the central vein 55 and are separated by sinusoid-like vascular endothelial lining cells. The central vein 55 is formed by one layer of endothelial cells, and open to those sinusoid-like vascular endothelial lining cells, as so-called hepatic sinusoids. The hepatocytes radiating from the central vein are organized as a hepatic plate, and the space between the anastomosing plates of the hepatocytes 60 are filled with liver sinusoid endothelial cells 65 receiving blood from the central vein. Portal triads 70, including portal vein, hepatic artery, and bile duct, are sinusoidally arranged at each of the corner of the classic hepatic lobule.

The human liver tissue are more like regularly branching and interconnecting sheets consisting of hepatocytes 60 and liver sinusoid endothelial cells 65 which radiate out from the center This architecture enlarges total contact area in the cellular level and enables the direct cell-to-cell contact between heterogeneous cells in particularly spatial orientation is also essential for normal development and organogensis. In addition, the vessels network provides for the exchange of substances between the blood and the liver, such as nutrition, oxygen, drug to be detoxificated, or glucose to be stored as glycogen.

Cell-based tissue cultivating techniques applied on artificial liver tissue are categorized as follows.

1. Liver X 2000 System. The system consisting of perfusing the medium through a hollow fabric module containing porcine hepatocytes and providing blood to flow by the fabric module. Porcine hepatocytes contained in the hollow fabric module are viable for only several ten days, and then those hepatocytes would lose activity and be gradually deteriorated with time. Porcine hepatocytes will survive longer if they are cultured in a microcapsule, which is made of algae and compatible with hepatocytes, instead of fabric module. However, only hepatocytes are aggregated in this artificial liver, so that direct cell-to-cell contact between heterogeneous cells for the rapid exchange of substances is absent. The artificial liver cultured by Liver X 2000 system could not function like a normal one.

2. Cell Co-culture System. The system consisting of putting two kinds of cells in the same petri dish and culturing them. Since cells are randomly arranged, cells could not be lined to form a specific pattern, such as vascular, ruga, or ball. Passive Cellular patterning techniques, such as cell sheet engineering or cultivation of cells on chemically modified substrate, was recently proposed, but they construct large scale of simple pattern. It is still insufficient to adequately guide or place single one cell and distribute the heterogeneous cells to reconstruct complicated architectures of tissue.

3. Active cell patterning technique. For example, laser trap is capable of manipulating individual cell to generate cell patterns. However, some drawbacks for laser-writing cell patterning come up for lacking the capability not only to control multiple cells simultaneously but also to move cells rapidly, largely as a result of restricted area of the cell pattern arranged by laser trap. In addition, use of magnetic force for the manipulation of cells was reported. Cells to be manipulated by magnetic force would be stuck by or implanted into magnetic particles. But, magnetic particles might be toxic to cells, and time-consuming and invasive process would influence the cell viability.

To sum up, real cell patterns of human liver cannot be rebuilt so far, so that function of bioartifical liver is not like that of normal one.

In addition, dielectrophoresis (DEP) is a phenomenon caused by the induced dipole of the polarizable particles in the solution under non-uniform electric fields. FIG. 2 illustrating principles of dielectrophoresis. Two dielectric particles are polarized in the presence of electric field. If the non-uniform electric field is applied, these two particles undergo dielectrophoretic forces and exhibit dielectrophoretic activity. Highly polarized particle are attracted to region of stronger electric field, and slightly polarized particle are repulsed to the region of lesser electric field. Consequently, fields of a particular frequency can manipulate particles with great selectivity. DEP has been widely demonstrated with separation of two polarized microparticles of different with permittivity, such as metallic and semiconductive carbon nanotubes.

### SUMMARY OF THE INVENTION

The invention is directed to a method of arranging cells and an electrode array applied thereto, in which the electrode array is biased to generate an enhanced dielectrophoresis so as to allow cells to be arranged into a pattern. Patterned cells are subsequently cultivated on the substrate to reconstruct vivid bioartificial tissue.

According to a first aspect of the present invention, a method of arranging cells is provided. The method comprises: (a) applying a voltage to two electrodes so as to allow a plurality of cells suspended in a dielectrophoresis-manipulating buffer (DEP-manipulating buffer) to be driven to be arranged into a pattern; (b) replacing the DEP-manipulating buffer with a solution comprising calcium ion and magnesium ion which helps the patterned cells adhere to the substrate; and (c) replacing the solution comprising calcium ion and magnesium ion with a medium so as to allow the patterned cells to grow on the substrate.

According to a second aspect of the present invention, an electrode array is provided. The electrode array comprises a first set of electrode including a first electrode and second electrode. The first electrode has a plurality of first projections on the periphery thereof. The second electrode surrounds the first electrode with a space interposed therebetween. The second electrode has a plurality of second projections evenly disposed thereon and towards the first electrode.

According to a third aspect of the present invention, an electrode array adopted to a dielectrophoretic reaction for arranging a plurality of cells is provided. The electrode array comprises two first electrodes and a second electrode. The two first electrodes are disposed with a space, and each first electrode has a first projection respectively. The second electrode has a second projection and is disposed between the first electrodes. The two ends of the second projection are toward the first projections respectively.

The invention will become apparent from the following detailed description of the preferred but non-limiting embodiments. The following description is made with reference to the accompanying drawings.

### BRIEF DESCRIPTION OF THE DRAWINGS

FIG. 1 is schematically illustrating a cross sectional view of a classic hepatic lobule of human liver.

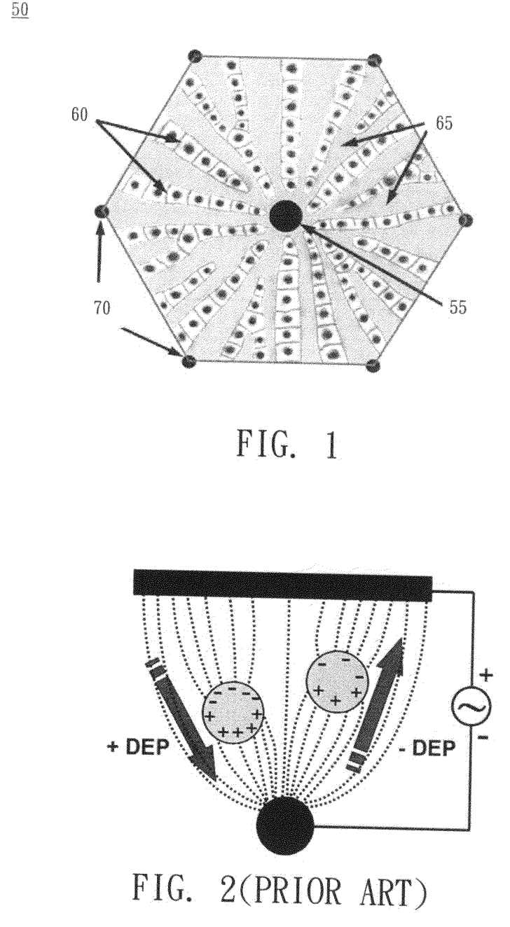

FIG. 2 illustrates principles of dielectrophoresis.

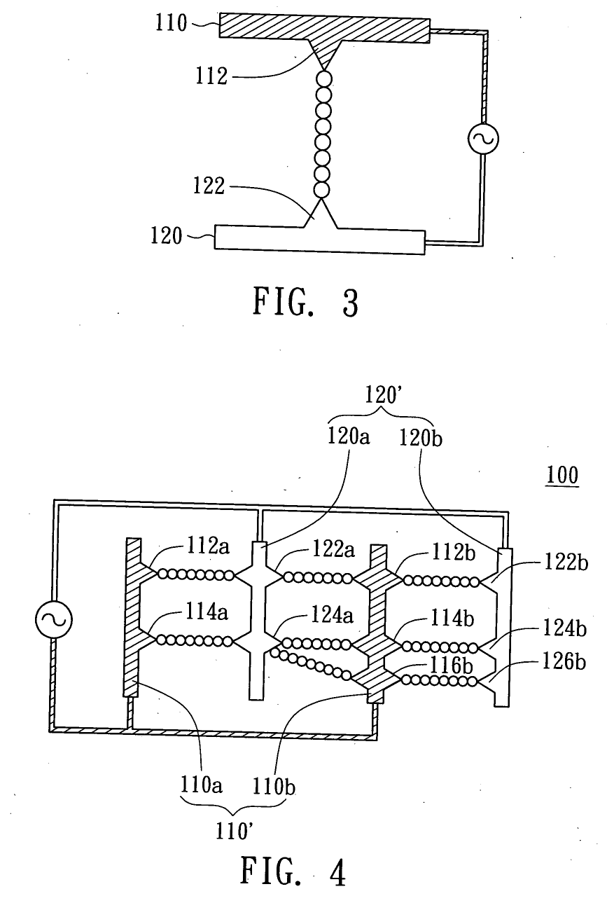

FIG. 3 schematically illustrates the unit of electrode array.

FIG. 4 schematically illustrates a pattern of an electrode array according to the first embodiment of the invention.

FIG. 5 schematically illustrates a pattern of an electrode array according to the second embodiment of the invention.

FIG. 6 schematically illustrates an electrode array according to the third embodiment of the invention.

FIGS. 7A-7D schematically illustrating the method of forming the electrode array of the third embodiment.

FIGS. 8A-8C schematically illustrates the method of arranging cells according to the third embodiment of the invention.

### DETAILED DESCRIPTION OF THE INVENTION

The invention is directed to a method of arranging cells, in which use of dielectrophoresis (DEP) with a specific electrode array for arranging cells into predetermined pattern is disclosed. Patterned cells are cultivated on the substrate to rebuild a bioartifical tissue, which vividly mimics real tissue.

The method of arranging cells includes applying a voltage to two electrodes so as to allow a plurality of cells suspended in a dielectrophoresis-manipulating buffer (DEP-manipulating buffer) to be driven to be arranged into a pattern; replacing the DEP-manipulating buffer with a solution comprising calcium ion and magnesium ion which helps the patterned cells adhere to the substrate; and replacing the solution comprising calcium ion and magnesium ion with a medium so as to allow the patterned cells to grow on the substrate.

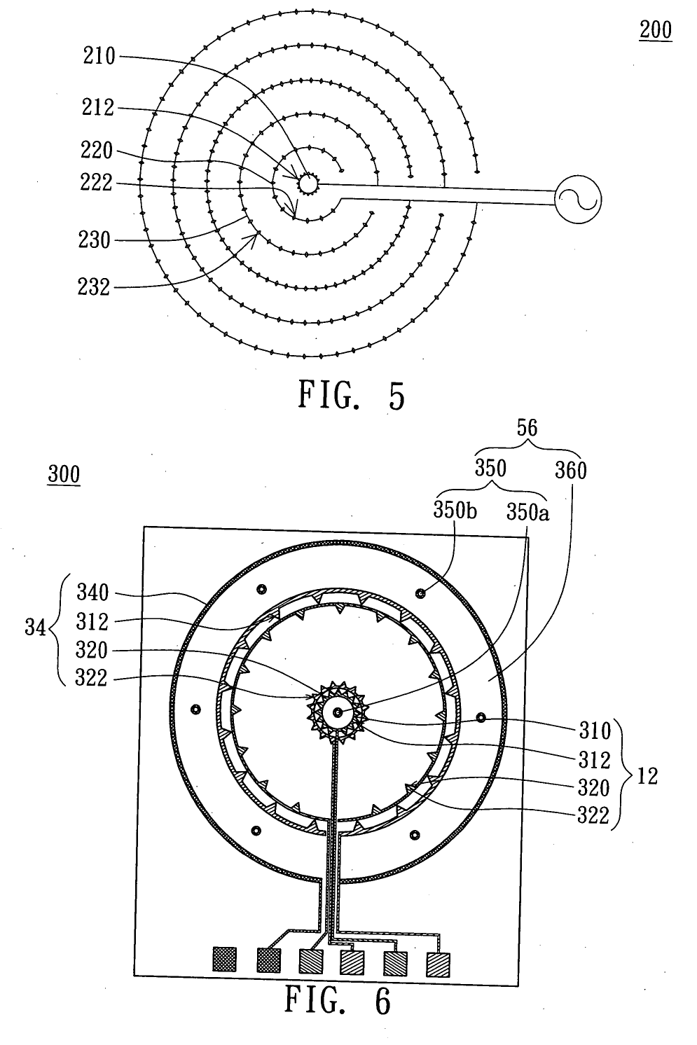

A unit of electrode array is proposed and widely applied in the following embodiments. Referring to FIG. 3, schematically illustrating the unit of electrode array. Two electrodes 110 and 120 respectively have two projections 1112 and 122. The shape of the projections 112 and 122 are with the angle of 30 to 75 degrees. When a voltage is applied on the two electrodes 110 and 120, an non-uniform electric field is generated and cells positioned therebetween are polarized. Simultaneously, polarized cells are attracted by the positive DEP force to the region of local electric-filed maximum, that is projections 112 and 122, and influenced by the interaction between polarized cells. It results in pearls-chained arrangement of the polarized cells between projection 112 and 122. Compared with conventionally DEP application for separating two kinds of cells, more precise manipulation of cells lined up between two predetermined points is achieved in the invention. Accordingly, various and complicated cell tissues could be reconstruct by method of the present invention associated with corresponding pattern of electrode array.

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

### First Embodiment

The method of arranging cells according to the first embodiment of the invention includes following steps. Firstly, a voltage is applied to two electrodes so as to allow a plurality of cells suspended in a dielectrophoresis-manipulating buffer (DEP-manipulating buffer) to be driven to be arranged into a pattern. In detail, a non-uniform electric field is generated to polarize cells when the voltage is applied, and then polarized cells are driven by a dielectrophoretic force. The DEP-manipulating buffer is an isotonic solution having the same concentration of solute as cells, and the permittivity of the DEP-manipulating buffer is less than that of the cells. Cells tends to be polarized more than the DEP-manipulating buffer, such that cells move to the region of local electric-filed maximum.

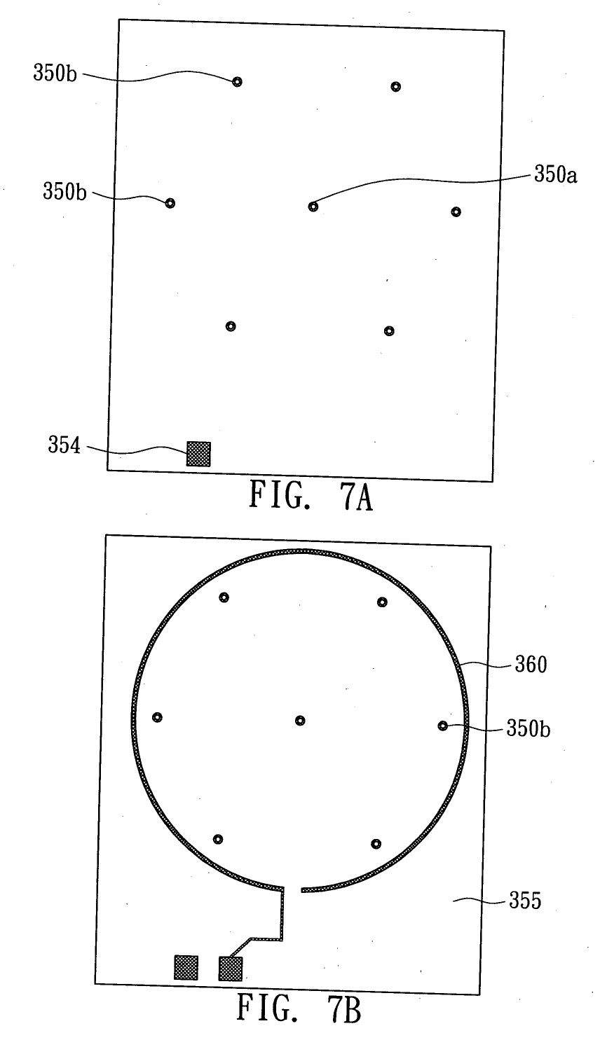

Cells, for example, could be arranged into a pattern of vessel or micro vessel net since they could be arranged as a straight or branched line according to the electrode array of the present embodiment. Referring to FIG. 4, schematically illustrating a pattern of an electrode array according to the first embodiment of the present invention. The electrode array includes a first electrode 110′ and a second electrode 120′. The first electrode 110′ includes two first conductors 110a and 110b electrically connected to each other. The second electrode 120′ includes two second conductors 120a and 120b electrically connected to each other. The first and second conductors 110a, 110b, 120a, and 120b are arranged in an alternating position, and each one of them has projection 112a, 112b, 122a, and 122b. When applying a voltage, cells arranged into a long line between projections 112a, 112b, 122a, and 122b to form a long line. The cell-chain could be elongated if more first and second conductors are incorporated into the first and second electrodes. Cells are less damaged under the condition of low voltage applied to a series of conductors than that of increased voltage applied to two electrodes at a distance

In addition, electrode array 100 further includes projection 114a, 124a, 114b, 116b, 124b, and 126b. The second conductor 120a has projections 124a, and the first conductor 110b adjacent thereto has two projection 114b and 116b. Cells are arranged between two projections, including projections 124a and 114b and projections 124a and 116b, so that cells form a branch at projections 124a. A net-like cell architecture, such as micro vessel net, could be reconstructed based on the electrode array disclosed above.

After cells are patterned, the DEP-manipulating buffer is replaced with a solution including calcium ion and magnesium ion. The solution preferably including 5 mM calcium ion and 5 mM magnesium ion, and it helps the patterned cells primarily adhere to the substrate or to adjacent cells.

Finally, the solution including calcium ion and magnesium ion is replaced with the medium so as to allow the patterned cells to grow on the substrate. Thus, the patterned cells could be cultivated.

### Second Embodiment

In this embodiment, cells could be arranged into a radiate pattern according to the electrode array of the present embodiment, and two kinds of cells are arranged into one pattern. The cell pattern, which mimics the hepatic lobule consisting of two different kinds of cells, is taken for an example to explicit the method of arranging cell and electrode array applied thereto of the present embodiment.

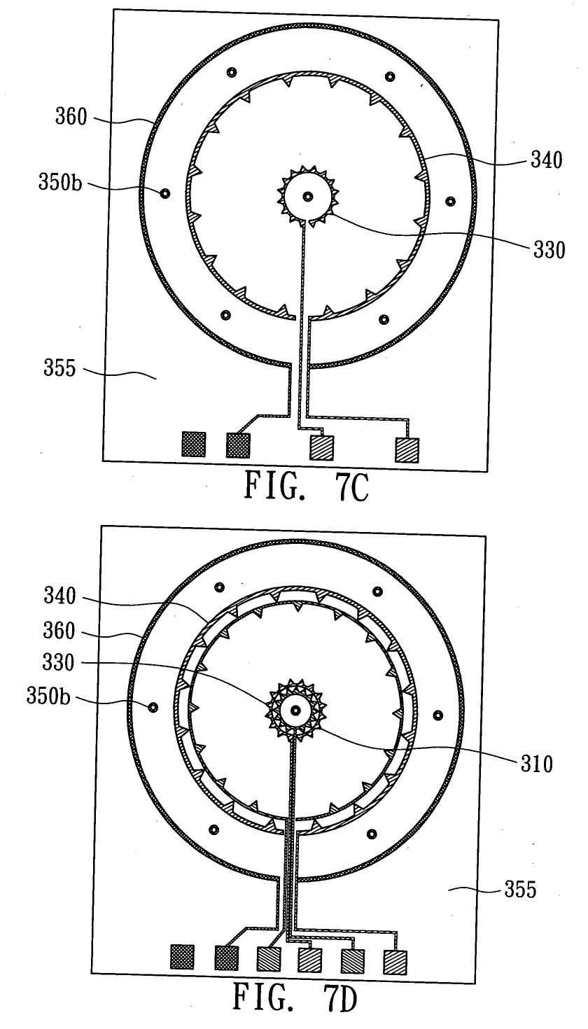

FIG. 5 schematically illustrates a pattern of an electrode array according to the second embodiment of the present invention. Referring to FIG. 5, the electrode array 200 includes a first electrode 210 and a second electrode 220. The first electrode 210 has many projections 212 on the periphery. The second electrode 220 surrounds the first electrode 210 with a space interposed between them. Many second projections 222 are evenly disposed on the second electrode 220 and toward the first electrode 210.

The space between the first projection 211 and the second projections 212 is ranged from about 80 to 100 micrometers (μm). The second electrode 220 is preferably an arc-shaped conductor, and the first electrode 210 is located at the center of the arc-shaped conductor. The second projections 222 appear along the second electrode 220 every π/8 radian angle.

The electrode array 200 also includes the third electrode 230, electrically connected to the first electrodes 210 and isolated from the second electrode 220. The third electrode 230 surrounds the second electrode 220 with a space of about 80 to 100 micrometers. Many third projections 232 are also evenly disposed on the third electrode 230 and toward the second electrode 220. In addition, the second projection 222 has two tips; one tip is positioned toward the first electrode 210, and the other tip is positioned toward the third electrode 230. Thus, cells would line up between the second projection 222 and the third projection 232.

The third electrode 230 preferably is an arc-shaped conductor, and the first electrode 210 is located at the center of the arc-shaped conductor. The third projections 232 appear along the third electrode 230 every π/16 radian angle.

Radiate cell pattern constructed by more cells can be achieved as long as the electrode array of the present embodiment is expanded to form a multiple concentric-ring electrode array. The first electrode 210 is at the center of the electrode array. The odd-order ring electrodes are electrically connected to the first electrode 210, and the even-order ring electrodes are electrically connected to the second electrode 220. Projections are disposed on the each electrode rings. The projections on the electrode would be distributed more densely if the electrode is positioned at the periphery of the electrode array. For example, projections of the electrode (i.e. fourth electrode) disposed outside the third electrode 230 appear along the electrode (i.e. fourth electrode) every π/32 radian angle

Referring to attachment 1, showing a simulation result for the root mean square of ac electric field (E_square) for the electrode array of the second embodiment when numerical simulation of DEP induced by applying the potentials of 5 Vpk-pk at 1 MHz is applied thereto. The tips of the first, second and third projections repeatedly provide numerous local gradient maximum of electric field (labeled as pink and red) when applying potentials to the first electrode 210 and the second electrode 220. Due to positive DEP effect, the cells, under appropriate ac potentials, could be guided from the lower electric-field region to the higher electric-field region. As a result, the cells could be attracted by the field induced DEP and from the precise radiate pattern. Proven by following experiments, the bioartificial tissue mimicking hepatic lobule, in which hepatocytes and liver sinusoid endothelial cells are arranged in an radiate pattern and alternative order, is achieved with use of the electrode array of the second embodiment.

Cell culture and medium are explicated as follows. Human liver cell line, HepG2 (ATCC, HB8065) and Human umbilical vein endothelial cells (HUVECs) are adopted to this experiment. Human liver cell line, HepG2 is maintained at 37° C. with 95%/5% air/CO₂ in Iscove's modified Dulbecco medium (IMDM, Gibco-BRL, NY) containing 10% (v/v) heat-inactivated fetal bovine serum (FBS, Biological Industries, Israel) and antibiotics (100 U/ml penicillin and 100 U/ml streptomycin, Sigma-Aldrich Co., MO). HUVECs are maintained in M200 medium supplemented with low serum growth supplement (LSGS). For observation of heterogeneous-cell patterning, HepG2 and HUVECs are pre-labeled with biocompatible fluorescent dyes, Dio (green) and Dil (red), for the identification at eh excitation/emission wavelengths of 488/520 nm and 530/565 nm, respectively.

The electrode array is formed on a glass substrate. The electrode array includes a Platinum layer of 2000 angstroms and a Titanium layer of 150 angstroms, and the Titanium layer helps the Platinum layer adhere to the glass substrate tightly. A poly-D-lysine film with positive charge is then coated on the substrate or electrode array to improve cell adhesion. Holes are mechanically punched through a polydimethylsiloxane (PDMS) top cover to form a chamber for the purpose of fluidic connections to outside tubing. After the oxygen plasma treatment on both the glass substrate and the PDMS top cover, these two parts are aligned and bond together.

Firstly, HepG2 cells suspended in the DEP-manipulating buffer (8.5% sucrose and 0.3% dextrose in ddH2O; conductivity: 10 ms/m) are positioned on the electrode array 200 under the condition of the applied CEP voltage of ac 5 Vpk-pk at 1 MHz. After the cells are arranged into a desire pattern, the inlet fluid is then switched to pure DEP-manipulating buffer without cells for 5 minutes to flush away the extra cells on the electrode region.

Next, the DEP-manipulating buffer supplemented with 5 mM calcium ion and 5 mM magnesium ion was injected to replace part of original DEP-manipulating buffer for 15 minutes to achieve stable sell0substrate adhesion for the following cell culture.

Afterward, the buffer in the chamber was replaced with IMDM medium (5 mM calcium ion and 5 mM magnesium ion) under the condition of a flow rate of 10 μl/min for 15 minutes. Primary cultivation of cells improves cell adhesion and viability. Referring to attachment 2 (a), showing distribution of HepG2 cells on electrode array of the second embodiment after DEP-manipulation. HepG2 cells are arranged in to radiate pattern and also aligned into the pearl-chain pattern between two opposite projections. Patterned HepG2 cells are going to be cultivated if one kind of cell needs to be arranged in this pattern.

HUVECs cells, the second kind of cells, are incorporated into the cell-pattern arranged in radiate and alternative order by filling the space between the cells HepG2 with HUVECs cells. HUVECs cells suspended in the DEP-manipulating buffer are also positioned on the electrode array 200 under the condition of the applied CEP voltage of ac 5 Vpk-pk at 1 MHz. Since the region of the local electric-field maximum has already been occupied by HepG2 cells, HUVECs cells are attracted to the available region of local electric-field maximum, that is, the region between the pearl-chained HepG2 cells. Afterward, HUVECs cells are adhered to the substrate in the solution comprising 5 mM calcium ion and 5 mM magnesium ion, and co-cultivated with cells HepG2 by M200 medium. Finally, patterned HepG2 cells and HUVECs cells are cultivated in the incubation.

For observation of heterogeneous-cell patterning, HepG2 and HUVECs are pre-labeled with biocompatible fluorescent dyes, Dio (green) and Dil (red), for the identification at eh excitation/emission wavelengths of 488/520 nm and 530/565 nm, respectively. Referring to attachment 2 (b) and (c), (b) shows the distribution of HepG2 cells and HUVECs cells on the electrode pattern after DEP manipulation, (c) is the experimental control group. HUVECs cells are, snared and filled into the left vacancy to form the additional alternate radiate pearl-chain array.

### Third Embodiment

In this embodiment, more than one set of electrode is combined in the electrode array for manipulating more than one kind of cells. It allows to construct more complicated and vivid bioartifical tissue. The cell pattern of human hepatic lobule is taken for an example to explicit the method of arranging cell and electrode array applied thereto of the present embodiment.

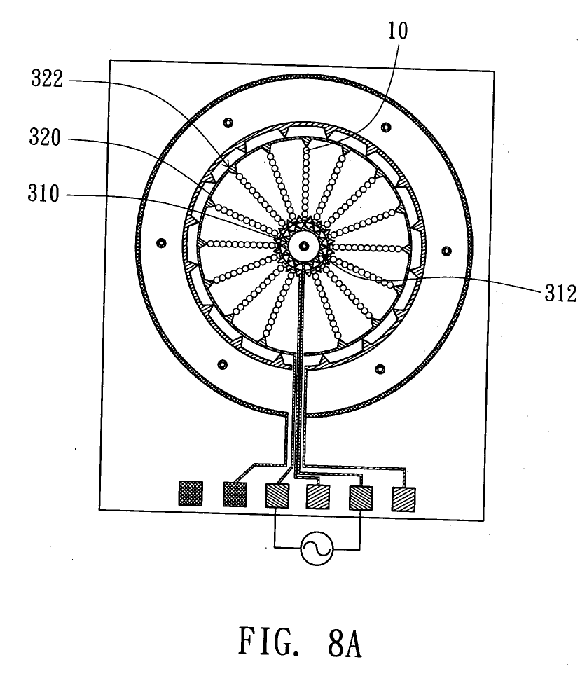

FIG. 6 schematically illustrates an electrode array according to the third embodiment of the present invention. Referring to FIG. 6, the electrode array 300 includes a first set of electrode 12, a second set of electrode 34, and a third set of the electrode 56, corresponding to the respective part of the hepatic lobule.

The first set of electrode 12 includes the first electrode 310 and the second electrode 320. Many first projections 312 are disposed on the periphery of the first electrode 310. The second electrode 320 surrounds the first electrode 310 with a space interposed between them. Many second projections 322 are disposed evenly on the second electrode 320 and toward the first electrode 310.

The second set of the electrode 34 is disposed between but disconnected to the first set of electrode 12. The second set pf electrode 34 includes the third electrode 330 and the fourth electrode 340. The third electrode 330 is adjacent to but disconnected to the first electrode 310, and many third projections are evenly disposed in the third electrode 330. The first projections 312 and third projections 332 are alternatively toward the second electrode 320. The fourth electrode 340 is adjacent to but disconnected to the second electrode 320, and is spaced from the third electrode 320. Many fourth projections 342 are toward the third electrode 330, and the fourth projections 342 and second projections 322 are alternatively toward the first electrode 310.

The third set of electrode 56 includes the fifth electrode 350 and the sixth electrode 360. The fifth electrode 350 has several conductors electrically connected to each other. One of the conductors 350a is located at the center of the first electrode 310, and rest of the conductors 350b are evenly distributed outside the second electrode 320. These conductors 350a and 350b are preferably annular. The sixth electrode 360 surrounds the fifth electrode 350.

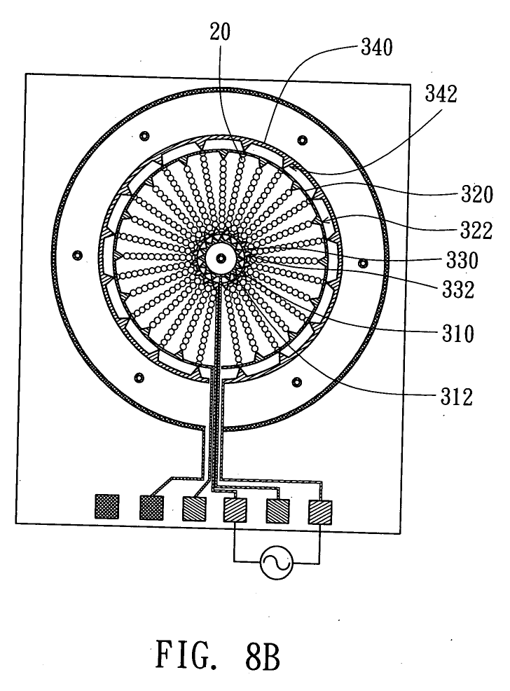

These three sets of the electrode may be fabricated in various way, and one of them is proposed and explicated with drawings as follows. Referring to FIGS. 7A˜7D, schematically illustrating the method of forming the electrode array of the third embodiment. Firstly, a thin conductive layer, such as aluminum layer of 2000 angstroms, is coated on the substrate, and an insulating layer 355 is formed thereon. Part of the insulating layer 355 is etched away via photolithography process, so that several annular conductors 350a and 350b are defined, as shown in FIG. 7A. If a voltage is applied to the pad 354, the potential will be conducted to all conductors 350a and 350b since they are made of the same conductive layer. The fifth electrode 350 consists of conductors 350a and 350b which are separated from and electrically connected to each other.

Arc-shaped metallic layer, i.e. 2000 Å aluminum, is then micromachined by the photolithography process with the E-gun evaporation and lift-off process, and the sixth electrode 360 has been formed as shown in FIG. 7B. Next, the third electrode 330 and the fourth electrode 340 are formed on the insulating layer 355 by photolithography process with the E-gun evaporation and lift-off process, as shown in FIG, 7C. The first electrode 310 and the second electrode 320 are formed by similar process, as shown in FIG. 7D.

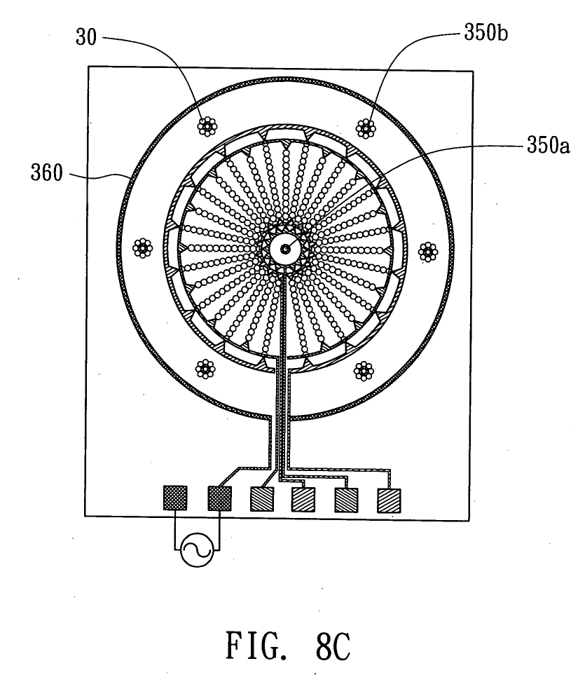

The step of arranging the second kind of cells in the method of arranging cells according to the third embodiment of the present invention is mainly different from that of embodiment above. The rest of steps that are similar to the above disclosure will not be repeated. Referring to FIG. 8A˜8C, schematically illustrating the method of arranging cells according to the third embodiment of the invention. The method to which the electrode array 300 is applied includes following steps. Firstly; a voltage is applied to the first electrode 310 and the second electrode 320, and the first kind of cells i.e. hepatocytes 10 suspended in the DEP-manipulating buffer are driven by the positive DEP effect and aligned into pearl-chain patter between the first projection 312 and second projection 322. The first kind of cells therefore are arranged into a radiate pattern as shown in FIG. 8A, and then primarily adhere on the substrate under the condition of suspension in solution comprising 5 mM calcium ion and 5 mM Magnesium.

Next, the second kind of cells 20, i.e. liver sinusoid endothelial cells, are suspended in the DEP-manipulating buffer, and the second set of electrode 34, including the third electrode 330 and the fourth electrode 340, is then biased. Cells 20 are arranged into another pattern according to the second set of electrode. The second kind of cells are driven by the positive DEP effect aligned into pearl-chain pattern between the third projections 332 and the fourth projections 342, as shown in FIG. 8B. The patterned cells 10 will not be interrupted by DEP-force or flowing fluid since they are primary adhered on the substrate. Afterward, the DEP-manipulating solution is replaced with the solution comprising calcium ion and magnesium ion so as to allow the patterned cells 20 to be adhered on the substrate. Finally, the previous solution is replaced with the medium, so that the patterned cells 10 and 20 are co-cultivated on the substrate.

It noteworthy that the second kind of cells 20, i.e. liver sinusoid endothelial cells, aligned between the third projection 332 and the fourth projection 342 are snared and filled in the to left vacancy between the first kind of cells 10, i.e. hepatocytes, aligned between the first projections 312 and the second projection 322. These two kinds of cells are arranged in radiate and alternate order, that is, every pearl-chain liver sinusoid endothelial cells are in contact with every pearl-chain of hepatocyes. That exactly mimics the shaped and even the function of real hepatic lobule.

Finally, the third set of electrode 56, including the fifth electrode 350 and the sixth electrode 360, is biased, so that the third kind of cells 30, i.e. liver sinusoid endothelial cells, are arranged into another pattern in similar way, as shown in FIG. 8C. The patterned cells 10 and 20 will not be interrupted by DEP-force or flowing fluid since they are primary adhered on the substrate. The third kind of cell 30 are driven by the positive DEP effect and attracted to the conductors 350a and 350b so as to form several annular pattern. Cells 30 aggregated at the conductor 350a mimic the central vein in the hepatic lobule, and cells 30 aggregated at the conductor 350b mimic the portal triads located at each corner of the hepatic lobule.

Vivid bioartifical liver tissue provides reliable platform for fundamental research of liver toxicity for drug application and liver metabolic function in vitro.

More than one set of electrode are incorporated in the electrode array of the present embodiment, so that more complicated pattern consisting of various can be achieved by repeating the steps of arrangement, adhesion, and primary cultivation. By doing so, cells can be arranged at the predetermined position since every arrangement is controlled by one set of electrode.

As described hereinafter, the method of arranging cells and the electrode array applied thereto have following advantages.

(1) High-resolution cell patterning technique is achieved by enhanced dielectrophoresis and the electrode array. The method, including precise arrangement of cells and subsequent cultivation of patterned cells, is capable of reconstructing various and complicated bioartificial tissue.

(2) High viability has been observed. The live and the dead cells can be monitored and distinguished simultaneously via in-situ fluorescence-staining method (i.e. FDA/EtBr cell-viability assay). After the above DEP operation under the same condition stated above in the second embodiment, the FDA/EtBr dyes are injected into the chamber. Referring to attachment 3, showing a microscope image for in-situ FDA/EtBr cell-viability assay. It shows both the viable cells (stained with green fluorescence) and the dead cells (stained with red fluorescence). The high-percentage cell survival rate of above 95% is observed for DEP-manipulation operation under the same condition stated above in the second embodiment.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of arranging cells, comprising:
applying a voltage to two electrodes so as to allow a plurality of cells suspended in a dielectrophoresis-manipulating buffer (DEP-manipulating buffer) to be driven to be arranged into a pattern;
replacing the DEP-manipulating buffer with a solution comprising calcium ion and magnesium ion which helps the patterned cells adhere to the substrate; and
replacing the solution comprising calcium ion and magnesium ion with a medium so as to allow the patterned cells to grow on the substrate.

2. The method according to claim 1, wherein the permittivity of the DEP-manipulating buffer and the solution comprising calcium ion and magnetic ion is less than that of the medium.

3. The method according to claim 1, wherein before the cells are arranged into a pattern, the method further comprises:
forming a film comprising poly-D-lysine on the substrate.

4. The method according to claim 1, wherein the DEP-manipulating buffer is an isotonic solution, and the permittivity of the DEP-manipulating buffer is less than that of the cells.

5. The method according to claim 1, wherein the solution comprises 5 mM calcium ion and 5 mM magnesium ion.

6. The method according to claim 1, wherein the two electrodes have two projections respectively, and the cells are lined up between the two projections when the voltage is applied to the two electrodes.

7. The method according to claim 6, wherein the shape of projections are with the angle of 30 to 75 degrees.

8. The method according to claim 1, wherein the two electrodes are a first electrode and a second electrode, the first electrode comprising two first conductors electrically connected to each other, the second electrode comprising two second conductors electrically connected to each other, the first and second conductors are substantially staggered;
wherein every first and second conductors has a tip, the cells are lined up between the projections when the voltage is applied on the first and second electrodes.

9. The method according to claim 1, wherein the two electrodes are a first electrode and a second electrode, a plurality of first projections disposed on the periphery of first electrode, the second electrode surrounding the first electrode with a space interposed therebetween, a plurality of projections evenly disposed on the second electrode and towards the first electrode;
wherein the cells are arranged as a radiate pearl-chain pattern when the voltage is applied on the first and second electrodes.

10. The method according to claim 1, wherein after the cells grow on the substrate, the method further comprises:
filling the space between the cells with a plurality of another cells.

11. The method according claim 1, wherein after the cells grow on the substrate, the method further comprises:
applying a voltage to another two electrodes so as to allow a plurality of another cells suspended in a dielectrophoresis-manipulating buffer (DEP-manipulating buffer) to be driven to be arranged into another pattern;
replacing the DEP-manipulating buffer with a solution comprising calcium ion and magnesium ion which helps the another patterned cells adhere to the substrate; and
replacing the solution comprising calcium ion and magnesium ion with a medium so as to allow the patterned cells and the another patterned cells to be co-cultivated on the substrate.

12. An electrode array, adopted to a dielectrophoretic reaction for arranging a plurality of cells, the electrode array comprising:
a first set of electrode, comprising:
a first electrode having a plurality of first projections on the periphery thereof;
a second electrode surrounding the first electrode with a space interposed therebetween, the second electrode having a plurality of second projections evenly disposed thereon and towards the first electrode.

13. The electrode array according to claim 12, wherein the space between the first and second projections is ranged from about 80 to 100 micrometers (μm).

14. The electrode array according to claim 12, wherein the second electrode is an arc-shaped conductor, and the first electrode is located at the center of the arc-shaped conductor, wherein the second projections appear along the second electrode every π/8 radian angle.

15. The electrode array according to claim 12, wherein the first set of electrode further comprising:
a third electrode electrically connected to the first electrode and isolated from the second electrode, the third electrode surrounding the second electrode with a space interposed therebetween, the third electrode having a plurality of third projections evenly disposed thereon and toward the second electrode;
wherein the second projection has two tips, one tips disposed toward the first electrode, and the other tips are disposed toward the third electrode.

16. The electrode array according to claim 15, wherein the third electrode is an arc-shaped conductor, and the first electrode is located at the center of the arc-shaped conductor, wherein the third projections appear along the third electrode every π/16 radian angle.

17. The electrode array according to claim 12, wherein the first set of electrode further comprises:
a fourth electrode electrically connected to the second electrode and isolated from the first electrode, the fourth electrode surrounding the third with a space interposed therebetween, the fourth electrode has a plurality of fourth projections evenly disposed thereon and toward the third electrode.

18. The electrode array according to claim 17, wherein the fourth electrode is an arc-shaped conductor, and the first electrode is located at the center of the arc-shaped conductor, wherein the fourth projections appear along the fourth electrode every π/32 radian angle.

19. The electrode array according to claim 12 further comprising a second set of electrode disposed between the first set of electrode and disconnected thereto, the second set of the electrode comprising:
a third electrode adjacent to and disconnected to the first electrode, a plurality of third projections evenly disposed in the third electrode; and
a fourth electrode adjacent to and disconnected to the second electrode, the fourth electrode spaced from the third electrode and having a plurality of fourth projections toward the third electrode;
wherein the first and third projections are alternatively toward the second electrode, and the fourth and second projections are alternatively toward the first electrode.

20. The electrode array according to claim 12 further comprising a second set of electrode, the second set of electrode comprising:
a third electrode having a plurality of conductors electrically connected to each other, one of the conductors located at the center of the first electrode, and rest of the conductors evenly distributed outside the second electrode; and
a fourth electrode surrounding the third electrode.

21. The electrode array according to claim 20, wherein the conductors are a plurality of annular conductors.

22. The electrode array according to claim 12 further comprising:
an adhesive layer comprising Titanium and formed on a substrate; and
a conductive layer comprising Platinum and formed on the adhesive layer.

23. An electrode array adopted to a dielectrophoretic reaction for arranging a plurality of cells, the electrode array comprising:
two first electrodes disposed with a space, each first electrode having a first projection respectively; and
a second electrode having a second projection and disposed between the first electrodes, two ends of the second projection are toward the first projections respectively.

24. The electrode array according to claim 23 further comprising another second electrode disposed outside the first electrodes so as to allow the first and second electrodes to be arranged in an alternate position.
